(19) United States
(12) Patent Application Publication
(10) Pub. No.: US 2006/0161059 A1
(43) Pub. Date: Jul. 20, 2006

(54) Variable geometry reference array

(75) Inventor: Jacque R. Wilson, Fort Wayne, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/039,040

(22) Filed: Jan. 20, 2005

(51) Int. Cl. A61B 5/05

(52) U.S. Cl. 600/424

(57) ABSTRACT

A tracking array for attachment to an object in a surgical environment includes tracking elements detectable by a surgical navigation system to permit tracking of the object.

### FIELD OF THE INVENTION

The present invention relates to devices used with surgical navigation systems. In particular, the present invention relates to an improved array of tracking elements attachable to an object to permit the surgical navigation system to track the position and orientation of the object during a surgical procedure.

### BACKGROUND

Many surgical procedures are now performed with surgical navigation systems in which sensors detect tracking elements attached in known relationship to an object in the surgical suite such as a surgical instrument, implant, or patient body part. The sensor information is fed to a computer that then triangulates the position of the tracking elements within the surgical navigation system coordinate system. Thus the computer can resolve the position and orientation of the object and display the position and orientation for surgeon guidance. For example, the position and orientation can be shown superimposed on and aligned with an image of the patient's anatomy obtained via traditional X-ray, CT scan, MRI, ultrasound, or other imaging technology.

It is desirable for the surgical navigation system to be able to recognize a particular tracking array and associate it with a particular object being tracked. Typically a different tracking array having a unique spatial pattern of tracking elements is provided for each different object to be tracked. For example, a supplier of surgical instruments to be used with surgical navigation procedures will provide a unique tracking array having a predetermined association with a particular instrument in the surgical navigation system software.

### SUMMARY

The present invention provides a tracking array for attachment to an object to facilitate tracking of the object by a surgical navigation system.

In one aspect of the invention, a surgical navigation array includes a body, a plurality of tracking elements mounted on the body, means for adjusting the relative position of the tracking elements to produce alternate spatial arrays of tracking elements.

In another aspect of the invention, a surgical navigation system includes means for tracking the position of an array of tracking elements and an array. The array includes an array body and a plurality of tracking elements mounted to the array body. At least one of the tracking elements is mounted to the array body by an adjustable tracking element mount. The adjustable tracking element mount is adjustable to independently change both the radial position and the angular position of the tracking element relative to the body.

In another aspect of the invention, a method includes: providing means for tracking the position of an array of tracking elements; providing an first array including an array body and a plurality of tracking elements mounted to the array body; and adjusting the relative position of the tracking elements of the first array to produce a first spatial arrangement of tracking elements.

### BRIEF DESCRIPTION OF THE DRAWINGS

Various examples of the present invention will be discussed with reference to the appended drawings. These drawings depict only an illustrative example of the invention and are not to be considered limiting of its scope.

FIG. 1 is a top plan view of an illustrative tracking array according to the present invention mounted on a surgical instrument;

FIG. 2 is a cross sectional view of the tracking array of FIG. 1 taken along line 2-2 of FIG. 1; and

FIG. 3 is a side elevation view of the tracking array of FIG. 1.

### DESCRIPTION OF THE ILLUSTRATIVE EXAMPLES

Embodiments of a tracking array for use with a surgical navigation system include an array body and tracking elements attached to the array body. The tracking elements are detectable by the surgical navigation system such that the three dimensional position of the tracking elements can be related to a surgical navigation coordinate system. For example, the surgical navigation system may include multiple sensors at known locations that feed tracking element position information to a computer. The computer may then use the position information from the multiple sensors to triangulate the position of each tracking element within the surgical navigation coordinate system. The tracking array may be attached to an object such as a surgical instrument, implant, or patient body part in a known orientation. The surgical navigation system can then determine the position and orientation of the object by detecting the position and orientation of the tracking array and then resolving the position and orientation of the object from the known relationship between the tracking array and the object.

The tracking elements may be detectable by imaging, acoustically, electromagnetically, and/or by other suitable detection means. Furthermore, the tracking elements may be active or passive. Examples of active tracking elements may include light emitting diodes in an imaging system, ultrasonic emitters in an acoustic system, and electromagnetic field emitters in an electromagnetic system. Examples of passive tracking elements may include elements with reflective surfaces.

A single tracking element may identify a point within the surgical navigation coordinate system. A pair of tracking elements may identify a line within the surgical navigation coordinate system. Three tracking elements may identify a plane within the surgical navigation coordinate system. With three tracking elements, the position and orientation of an object connected to the tracking array may be determined. By providing more than three tracking elements, redundant identification of a plane within the surgical navigation coordinate system is possible so that if one of the redundant tracking elements should become momentarily blocked from detection, the surgical navigation system may continue to track the tracking array and associated object. The surgical navigation system is configured to track a predetermined number of tracking elements for each array, including redundant tracking elements if present.

The tracking array may have the tracking elements arranged in a predetermined spatial arrangement that not only permits tracking of the array but also identification of a particular array by detecting the predetermined spatial arrangement. The tracking array may be a variable geometry array having an array body with tracking elements repositionable in different unique spatial arrangements. Thus, the one array body geometry can be produced inexpensively in large quantities and used to provide individually unique array configurations. Similarly, the end user realizes cost and space savings since they need only stock one array that can be reconfigured for use with multiple objects to be tracked. The tracking elements may be repositioned relative to the array body to create alternate spatial configurations by changing the radial and/or angular positions of the tracking elements to create uniquely identifiable tracking arrays from the same array body and tracking elements. The tracking elements may be continuously adjustable or they may be adjustable in predetermined steps. A locking mechanism may be provided to lock the tracking elements in position.

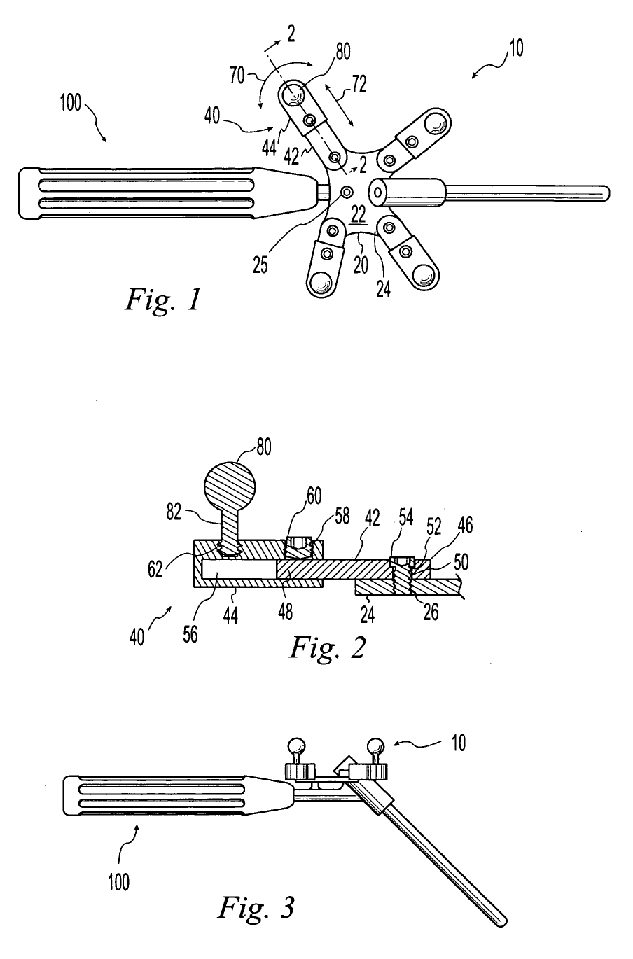

FIGS. 1-3 depict an illustrative variable geometry array 10 having a body 20 and adjustable mounting mechanisms 40 for connecting tracking elements 80 to the body 20. The body 20 is a flat plate having a palmate layout including a central portion 22 and a plurality of lobes 24. The body 20 may be machined, molded, or otherwise formed. The body 20 may be made of metal, polymers, and/or other suitable materials. The body 20 includes a mounting screw 25 passing through a bore in the body.

Each adjustable mounting mechanism 40 includes a pivot arm 42 and a slider 44. The pivot arm 42 includes a pivot end 46 and a slider engaging end 48 (FIG. 2). The pivot end 46 includes a transverse through bore 50 having a shoulder 52 for receiving a pivot screw 54. The pivot screw 54 threads into a threaded bore 26 in one of the lobes 24 of the body 20 to form a pivot about which the pivot arm 42 may be rotated as indicated by arrow 70. The pivot screw 54 may be tightened to lock the pivot arm 42 in a particular angular orientation relative to the body 20.

The slider 44 includes a non-circular opening 56 for receiving the slider engaging end 48 of the pivot arm 42. The non-circular engagement of the slider 44 and pivot arm 42 constrains the slider 44 to linear translation as indicated by arrow 72. The slider 44 includes a threaded bore 58 communicating with the non-circular opening 56 to receive a locking screw 60. The locking screw 60 may be tightened against the pivot arm 42 to lock the position of the slider 44 relative to the pivot arm 42. The slider 44 includes a second threaded bore 62 for receiving a threaded mounting post 82 of a tracking element 80 to connect the tracking element 80 to the slider 44. Alternatively, the connection of the pivot arm 42 to the body 20 and the connection of the slider 44 to the pivot arm 42 may be accomplished with bolts, clamps, ratchets, and/or other suitable adjustable connection mechanisms. One or more of the array elements 80 are adjustable to create a unique spatial configuration.

The variable geometry tracking array 10 is mounted to a surgical component 100 by engaging the mounting screw 25 with the component 100. Alternatively, the array 10 may be mounted with bolts, dovetails, hook and loop material, adhesives, and/or other suitable mounting mechanisms.

In use, the variable geometry tracking array 10 is mounted to the surgical component 100. The array 10 elements 80 are adjusted to a spatial configuration associated with the particular surgical component. Each pivot arm 42 is rotated about its pivot screw 54 to a desired angular position and each slider 44 is slid along its pivot arm 42 to a desired radial position corresponding to a desired tracking element 80 position. Predetermined angular and radial positions may be marked on the adjustment mechanism to guide the user in positioning the tracking elements 80 in predefined spatial configurations. Alternatively, the assembly may be placed within the surgical navigation system environment and the surgical navigation system may be used to guide positioning the tracking elements 80. As the user moves the elements 80 angularly and radially, the surgical navigation system can give feedback indicating when the elements 80 are arranged in a predetermined recognizable pattern. The system can also be configured to give feedback as to specific adjustments required for each element 80 to produce a desired pattern. Furthermore, the tracking elements 80 may be adjusted into a random pattern by the user and then that pattern may be associated with a particular surgical component by the surgical navigation system. When multiple arrays 10 are to be used, the user can adjust subsequent arrays 10 into random patterns and the surgical navigation system can evaluate the pattern and indicate to the user if the pattern is distinguishable from the other array patterns in use. If the pattern is distinguishable by the system, then it is associated with a subsequent particular surgical component. If the pattern is not distinguishable, the system may prompt the user to readjust the array pattern.

Although embodiments of a tracking array and its use have been described and illustrated in detail, it is to be understood that the same is intended by way of illustration and example only and is not to be taken by way of limitation. Accordingly, variations in and modifications to the tracking array and its use will be apparent to those of ordinary skill in the art, and the following claims are intended to cover all such modifications and equivalents.

What is claimed is:

1. A surgical navigation array comprising:
a body;
a plurality of tracking elements mounted to the body; and
means for adjusting the relative position of the tracking elements to produce alternate spatial arrays of tracking elements.

2. The surgical navigation array of claim 1 wherein the means for adjusting comprises means for adjusting the radial position of at least one tracking element relative to the body.

3. The surgical navigation array of claim 1 wherein the means for adjusting comprises means for adjusting the angular position of at least one tracking element relative to the body.

4. The surgical navigation array of claim 1 wherein the means for adjusting comprises means for independently adjusting both the radial position and the angular position of at least one tracking element relative to the body.

5. The surgical navigation array of claim 1 wherein the means for adjusting comprises an adjustable mechanism for connecting at least one tracking element to the body, the mechanism including a pivot arm pivotably mounted to the body and a slider slidingly mounted to the pivot arm, the at least one tracking element being mounted on the slider.

6. A surgical navigation array comprising:
a body; and
a plurality of tracking elements mounted to the body, at least one tracking element being adjustable to produce alternate spatial arrays of tracking elements.

7. A surgical navigation system comprising:
means for tracking the position of an array of tracking elements; and
an array including an array body and a plurality of tracking elements mounted to the array body, at least one tracking element being mounted to the array body by a tracking element mount, the tracking element mount being adjustable to independently change both the radial position and the angular position of the tracking element relative to the body.

8. The surgical navigation system of claim 7 the tracking element mount is adjustable to change the tracking element position continuously.

9. The surgical navigation system of claim 7 the tracking element mount is adjustable to change the tracking element position in discrete steps.

10. A method comprising:
providing a means for tracking the position of an array of tracking elements;
providing a first array including an array body and a plurality of tracking elements mounted to the array body; and
adjusting the relative position of the tracking elements of the first array to produce a first spatial arrangement of tracking elements.

11. The method of claim 10 wherein the step of adjusting comprises tracking the tracking elements and generating feedback to indicate when the tracking elements are configured in a predetermined spatial arrangement.

12. The method of claim 10 wherein the step of adjusting comprises tracking the tracking elements and generating feedback to guide the adjusting to achieve a predetermined spatial arrangement.

13. The method of claim 10 further comprising:
storing the first spatial arrangement of tracking elements in a computer memory; and
associating the stored first spatial arrangement with a particular first object within the surgical navigation system coordinate system.

14. The method of claim 13 further comprising:
providing a second array including an array body and a plurality of tracking elements mounted to the array body;
adjusting the relative position of the tracking elements of the second array to produce a spatial arrangement of tracking elements;
evaluating the spatial arrangement of the second array to verify that it is distinguishable by the means for tracking from the first array;
storing the second spatial arrangement of tracking elements in a computer memory; and
associating the stored second spatial arrangement with a particular second object within the surgical navigation system coordinate system.

15. The method of claim 10 wherein the step of adjusting comprises independently changing both the radial position and the angular position of the tracking elements relative to the body.
